(19) United States
(12) Patent Application Publication
(10) Pub. No.: US 2025/0359927 A1
(43) Pub. Date: Nov. 27, 2025

(54) PULSE ENERGIZING DEVICE AND PROCESSING METHOD FOR THE SAME

(71) Applicant: SHENZHEN PULSECARE MEDICAL TECHNOLOGY CO., LTD., Shenzhen (CN)

(72) Inventor: Jianwen Tan, Shenzhen (CN)

(21) Appl. No.: 19/260,320

(22) Filed: Jul. 4, 2025

Related U.S. Application Data
(63) Continuation of application No. PCT/CN2025/085752, filed on Mar. 28, 2025.

(30) Foreign Application Priority Data
May 27, 2024 (CN) 202410660905.5

(51) Int. Cl. A61B 18/14; A61B 18/00

(52) U.S. Cl. CPC A61B 18/1492; A61B 2018/00267; A61B 2018/00351; A61B 2018/00577; A61B 2018/144; A61B 2018/1467

(57) ABSTRACT

A pulse energizing device includes a multi-cavity component, at least one insert component and a plurality of electrodes. The multi-cavity component is cut along an axial direction to form a plurality of sub-tube portions, and each electrode is mounted around a respective sub-tube portion. The insert component is inserted into the multi-cavity component, and one end of the insert component is connected with a distal end. Therefore, under the pull of the insert component, the distal end is movable along the axial direction to drive deformation of each sub-tube portion. In the present disclosure, by setting the multi-cavity component, and directly processing on the basis of the multi-cavity component, a structure and a processing method are simple, so that the pulse energizing device can be reproduced.

### CROSS-REFERENCE TO RELATED APPLICATIONS

The present disclosure is a Continuation Application of PCT Application No.PCT/CN2025/085752, filed on Mar. 28, 2025, which claims the priority of Chinese Patent Application No. 202410660905.5, filed on May 27, 2024, the entire contents of which are hereby incorporated by reference.

### TECHNICAL FIELD

The present disclosure relates to the technical field of medical devices, and particularly to a pulse energizing device and a processing method for the same.

### BACKGROUND

Atrial fibrillation is the most common arrhythmia, which may lead to stroke, cardiomyopathy, and the like, and may lead to death in severe cases. With the increase in age, the incidence of atrial fibrillation continues to rise. Percutaneous catheter ablation is a first-line treatment for atrial fibrillation and has been widely recognized. A purpose of ablation is to destroy potential arrhythmic myocardial tissue, prevent the propagation of abnormal electrical signals, or destroy the abnormal electrical signal conduction of cardiac tissue. Ablation therapy includes a plurality of aspects: one aspect is thermal ablation, such as radiofrequency ablation, laser ablation, microwave ablation, and the like, and the other aspect is pulsed ablation using the principle of bioelectroporation.

### SUMMARY

In some embodiments, the present disclosure provides a pulse energizing device, including: a multi-cavity component, defining a central cavity and a plurality of peripheral cavities surrounding the central cavity; the plurality of peripheral cavities and the central cavity extend along an axial direction without communicating with each other; a wall thickness of the multi-cavity component is greater than a preset value; a portion of the multi-cavity component between a proximal end and a distal end thereof in the axial direction is cut along the axial direction into a plurality of sub-tube portions that are separated in a circumferential direction; each of the plurality of sub-tube portions is provided with a respective peripheral cavity therein, and each of the plurality of sub-tube portions is provided with a through hole communicating with the corresponding peripheral cavity therein; and the distal end of the multi-cavity component is movable along the axial direction of the multi-cavity component, to drive the plurality of sub-tube portions to transform between one configuration of extending along a straight line and another configuration of protruding outward in a curved shape; at least one insert component, one end of the at least one insert component extending through the central cavity to connect with the distal end of the multi-cavity component; and a plurality of electrodes, each of the plurality of electrodes being mounted around a respective sub-tube portion and configured to deliver a pulse current.

In some embodiments, the present disclosure further provides a processing method for processing the pulse energizing device described above, and the processing method includes: inserting a positioning pin into a central cavity of a multi-cavity component to be processed, and inserting a plurality of core rods into a plurality of peripheral cavities respectively; placing and fixing the multi-cavity component to be processed on a processing tooling, and bringing a cutting blade on the processing tooling to abut against a portion of the multi-cavity component to be cut; driving the multi-cavity component or the cutting blade on the processing tooling to move, causing the cutting blade to cut the multi-cavity component to form the plurality of the sub-tube portions; and removing the multi-cavity component from the processing tooling, and pulling out the positioning pin and each core rod respectively.

### BRIEF DESCRIPTION OF DRAWINGS

In order to more clearly illustrate the technical solutions in the embodiments of the present disclosure, a brief introduction to drawings described in the embodiments is as follows. Apparently, the following drawings are merely some embodiments of the present disclosure, for those skilled in the art, other drawings can be obtained based on these drawings without creative labor.

FIG. 1 is a structural schematic view of a pulse energizing device according to one embodiment of the present disclosure.

FIG. 2 is another structural schematic view of the pulse energizing device according to one embodiment of the present disclosure.

FIG. 3 is a cross-sectional schematic view of a multi-cavity component adjacent to a proximal end according to one embodiment of the present disclosure.

FIG. 4 is a cross-sectional schematic view of an insulated electrical lead according to one embodiment of the present disclosure.

FIG. 5 is a cross-sectional schematic view of an electrode according to one embodiment of the present disclosure.

FIG. 6 is a schematic view of an electrode provided with a voltage balancing structure according to one embodiment of the present disclosure.

FIG. 7 is a schematic view of an electrode provided with a voltage balancing ring according to one embodiment of the present disclosure.

FIG. 8 is a structural schematic view of a processing tooling according to one embodiment of the present disclosure.

FIG. 9 is a schematic view illustrating a multi-cavity component with a positioning pin and a core rod inserted therein according to one embodiment of the present disclosure.

### DESCRIPTION OF REFERENCE NUMERALS

1, pulse energizing device; 11, multi-cavity component; 101, proximal end; 102, distal end; 111, central cavity; 112, peripheral cavity; 113, sub-tube portion; 114, braided layer; 12, insert component; 13, electrode; 131, receiving hole; 132, voltage balancing structure; 133, voltage balancing ring; 14, insulated electrical lead; 141, conductive core; 142, insulating layer.
2, processing tooling; 21, workbench; 22, blade holder; 23, cutting blade; 24, positioning block; 25, positioning seat; 26, push rod; 27, positioning pin; 28, core rod.

### DESCRIPTION OF EMBODIMENTS

In order to make the purpose, technical solutions and advantages of the present disclosure more clearly understood, the present disclosure is further described in detail below in conjunction with accompanying drawings and embodiments. It should be understood that specific embodiments described herein are only used to explain the present disclosure and are not intended to limit the present disclosure.

Various specific technical features described in the various embodiments can be combined in any appropriate manner as long as there is no contradiction. For example, different embodiments and technical solutions can be formed by combining different specific technical features. In order to avoid unnecessary repetition, various possible combinations of the specific technical features in the present disclosure are not described separately.

In the following descriptions, the terms “first, second . . . ” are only used to distinguish different objects, and do not indicate that the objects have similarities or connections with each other. It should be noted that the orientation descriptions “above”, “below”, “outside”, “inside” and the like mentioned herein are all orientations in a normal use state. The “left” and “right” directions refer to the left and right directions shown in the corresponding schematic views, which may be or may not be the left and right directions in the normal use state.

It should be noted that the terms “include”, “comprise” or any other variants thereof are intended to cover a non-exclusive inclusion, therefore, a process, method, article or device that includes/comprises a series of elements is not limited to those elements, but also includes/comprises other elements not explicitly listed, or elements inherent to such process, method, article or device. Without further limitations, an element defined by the phrase “including/comprising one . . . ” does not exclude the presence of other identical elements in the process, method, article or device including/comprising the element. “Plurality” means a number greater than or equal to two.

Pulsed Field Ablation (PFA) is a novel tissue ablation method based on high-voltage pulsed energy, which has emerged in recent years and mainly uses a principle of irreversible electroporation (IRE). Through the action of a high-voltage pulsed electric field on cells, irreversible perforations are generated in cell membranes, thereby causing the cells to gradually necrose, to ultimately achieve a purpose of tissue ablation. Due to differences in tissue electrical properties and damage thresholds of cells to high-voltage pulsed energy, PFA exhibits excellent tissue selectivity. For example, myocardial tissue is more sensitive to high-voltage pulsed electric fields, while neural tissue has a higher tolerance to pulsed electric fields. Therefore, by selecting appropriate intensity of the high-voltage pulsed electric field, ablation of selective tissue, such as ablation of tissue near nerves and blood vessels, and the like, can be achieved. Except for the selective tissue mentioned above, PFA is generally considered as a non-thermal ablation technique. That is, no heat and no temperature increase in tissue occurs during an ablation process, thereby avoiding the heat sink effect associated with traditional ablation methods such as radiofrequency, microwave and cryoablation through irreversible electroporation. Therefore, PFA is considered to have superior advantages in ablation of temperature-sensitive tissues (such as tissues in proximity to the gallbladder, bile duct, esophagus and the like), especially when atrial fibrillation is treated by ablation, PFA has the advantages of short ablation duration and protection of tissues such as the treatment area or blood vessels.

When performing PFA ablation for atrial fibrillation, it is necessary to perform circumferential pulmonary vein ablation quickly and ensure the contact of catheter electrodes. Therefore, the design of PFA catheters is crucial. However, typical PFA catheters have complicated structures that are difficult to manufacture and process, which increases the difficulty of producing the PFA catheters. Embodiments of the present disclosure provide a pulse energizing device or catheter, and the pulse energizing device or catheter is generally connected to a control device and a high-voltage pulse generator. The purpose of tissue ablation is achieved by means of inserting one end of the pulse energizing device into a blood vessel, and advancing the pulse energizing device along the blood vessel into the tissue to be treated by operating the control device (such as a control handle), delivering a high-voltage, high-frequency pulse voltage generated by the high-voltage pulse generator to the pulse energizing device after the pulse emerging device has been delivered in place, thereby establishing a high-intensity electric field at the tissue to be treated, and forming an area with high current density, through the action of the high-voltage pulsed electric field on cells, irreversible perforations being generated in the cell membrane, thereby causing the cells to gradually necrose.

The term “electroporation” herein refers to the application of an electric field to cell membranes to change the permeability of the cell membranes to an extracellular environment. The term “irreversible electroporation” herein refers to the application of an electric field to cell membranes to permanently change the permeability of the cell membranes to the extracellular environment. For example, it can be observed that one or more pores are formed in the cell membranes when the cells are subjected to the irreversible electroporation, and the one or more pores remain after the electric field is removed. The term “proximal end” herein refers to one end of the pulse energizing device that is adjacent to an operator, or one end that is connected to an action of the operator. The term “distal end” herein refers to one end of the pulse energizing device that is inserted into the blood vessel, or one end that is adjacent to the tissue to be treated.

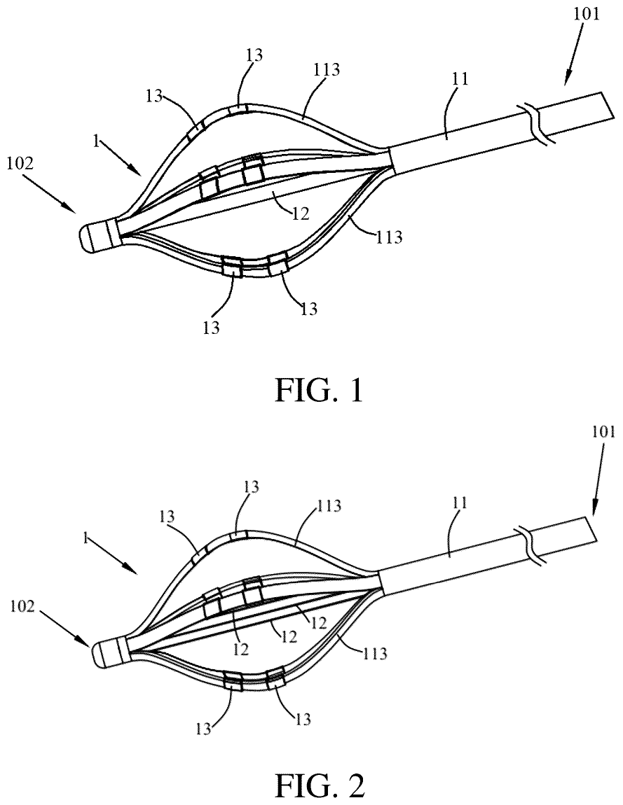

As shown in FIG. 1 to FIG. 3, a pulse energizing device 1 is provided by one embodiment of the present disclosure, including a multi-cavity component 11, one or more insert components 12, and a plurality of electrodes 13. The multi-cavity component 11 is generally a tubular material with a circular shaped cross-section, and is provided with a central cavity 111 and a plurality of peripheral cavities 112 surrounding the central cavity 111 therein. The peripheral cavities 112 and the central cavity 111 extend along an axial direction of the multi-cavity component 11 and are non-communicating with each other; for example, a length direction of each peripheral cavity 112 and the central cavity 111 itself extends substantially along a length direction of the multi-cavity component 11.

In some other embodiments, a wall of the multi-cavity component 11 has a thickness greater than a preset value, for example, 0.5-1.5 mm. The specific value of the thickness is suitable to ensure the reliable configuration of the central cavity 111 and each peripheral cavity 112, and avoid an oversized diameter which is not conducive to intravascular delivery.

In some embodiments, a portion of the multi-cavity component 11 between a proximal end 101 and a distal end 102 in the axial direction is cut into a plurality of sub-tube portions 113 along the axial direction, and the sub-tube portions 113 are arranged to separate apart in a circumferential direction of the multi-cavity component 11. A length direction of each sub-tube portion 113 is arranged along the length direction of the multi-cavity component 11, and each sub-tube portion 113 is provided with one peripheral cavity 112 therein. The distal end of the multi-cavity component 11 is axially movable, so that each sub-tube portion 113 can be transformed between one configuration of extending along a straight line and another configuration of protruding outward in a curved shape. For example, each sub-tube portion 113 possesses elastic deformability and can be bent under the action of an external force, and after the external force disappears, each sub-tube portion 113 can automatically recover to an initial state or substantially initial state under the action of its own elastic deformation force. Generally, the multi-cavity component 11 is made of a polymer material meeting medical usage standards, such as Pebax (thermoplastic nylon elastomer) and PA (polyamide), which have high strength, high fracture resistance, and excellent elasticity. The multi-cavity component 11 can be formed into a structure with different hardness at different positions with gradual transition. For example, the hardness gradually decreases from the distal end to the proximal end, so that the distal end has better insertion performance, which facilitates to guiding movement in the blood vessel, while the hardness gradually decreases as approaching the proximal end, which is convenient for bending adjustment. The hardness of a middle region of the multi-cavity component 11 is moderate, for example, the hardness of the middle region of the multi-cavity component 11 is greater than the hardness of the proximal end of the multi-cavity component 11, to ensure the overall strength and pushability. Of course, the hardness of the multi-cavity component 11 can also be set to other distributions according to usage requirements, for example, the hardness of a certain region is designed to be higher or lower, which is flexible in design.

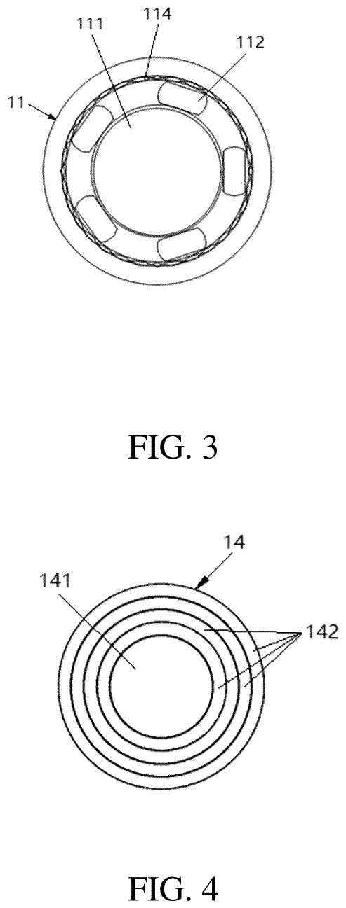

In some embodiments, as shown in FIG. 2 and FIG. 3, one end of the insert component 12 (for example, a distal end of the insert component 12) is inserted into the central cavity 111 and extends to connect with the distal end of the multi-cavity component 11. An opposite end of the insert component 12 (for example, a proximal end of the insert component 12) is connected to a control device or used for hand-held operation by an operator. In this way, an acting force can be transmitted to the distal end of the multi-cavity component 11 by pulling the insert component 12, so that the distal end of the multi-cavity component 11 can be moved toward a proximal end of the pulse energizing device 1, thereby causing each sub-tube portion 113 to be protruded outward and bent. Each electrode 13 is mounted around a respective sub-tube portion 113, to introduce a pulse current into the pulse energizing device 1. For example, the pulse current is introduced when the sub-tube portions 113 protrude outward in a curved shape or when the sub-tube portions 113 extend along a straight line, so that ablation treatment of tissue is performed when the sub-tube portions 113 protrude outwards in the curved shape or extend along the straight line. Therefore, in this state, the electrodes 13 provided on the respective sub-tube portions 113 discharge electricity to achieve a treatment purpose.

In some cases, under high-voltage and high-frequency pulses (for example nanosecond pulses or millisecond pulses), the electrodes 13 may be easy to contact with blood and generate a certain amount of heat, so that surface temperatures of the electrodes 13 increase to promote a coagulation mechanism of the blood around the electrodes 13, thereby resulting in scabs on surfaces of the electrodes 13. The scabs further increase the contact resistance between the electrodes 13 and the blood, resulting in a vicious cycle. In one embodiment of the present disclosure, a through hole (not shown in figures) communicating with the peripheral cavity 112 is defined on each sub-tube portion 113, and the through hole is arranged adjacent to the electrodes 13. Fluid (such as saline) is injected into each peripheral cavity 112, and then the injected fluid flows out from a corresponding through hole. Fluid flowing out from the vicinity of the electrodes 13 can not only improve the electrical conductivity of the surrounding area, but also play a role in continuously cooling the electrodes 13, thereby achieving a purpose of cooling the electrodes 13 and reducing the risk of scab formation.

In some other embodiments, the number of the through holes is 2 to 6, such as 4, and a plurality of through holes are spaced apart along the circumferential direction of the sub-tube portions 113.

The pulse energizing device 1 provided by the embodiment of the present disclosure includes the multi-cavity component 11, the insert component 12, and the electrodes 13. The multi-cavity component 11 is provided with the central cavity 111 and the peripheral cavities 112 surrounding the central cavity 111 therein. The portion between the proximal end and the distal end of the multi-cavity component 11 is cut axially to form the sub-tube portions 113 separated in the circumferential direction, one peripheral cavity 112 is provided in each sub-tube portion 113, and each sub-tube portion 113 is provided with the electrode 13. Moreover, the insert component 12 is inserted into the central cavity 111, and one end of the insert component 12 is connected to the distal end of the multi-cavity component 11, so that the distal end of the multi-cavity component 11 can move along the axial direction of the pulse energizing device 1 under the pull of the insert component 12, thereby driving the sub-tube portions 113 to transform between the two configurations of extending along the straight line and protruding outward in the curved shape. In some embodiments, when the sub-tube portions 113 are protruded outward in the curved shape or extended along the straight line, a pulse current is introduced to achieve a discharge, thereby achieving the purpose of tissue ablation. In the embodiment of the present disclosure, the multi-cavity component 11 is provided, and the processing is directly performed on the basis of the multi-cavity component 11. The multi-cavity component 11 itself has a simple structure, which can meet user's requirements, so that too many other complicated structural designs are not required, and thus a structure of the pulse energizing device 1 is simpler. In addition, the processing and production are achieved through cutting. The production process is relatively simple with low requirements, and can be reproduced repeatedly, which excellently meets the reproducibility requirements of the pulse energizing device 1.

In some embodiments, as shown in FIG. 1 and FIG. 2, the number of the sub-tube portions 113 formed by cutting the multi-cavity component 11 can be multiple, optionally 3 to 12, and the specific number of the sub-tube portions 113 may be set according to actual use requirements. In this way, in the same position, the sub-tube portions 113 are oriented toward different positions in the circumferential direction, so that discharge ablation can be performed on different parts of the tissue at the location, without the need of rotation in the same position to achieve treatment of different parts, thereby improving the convenience of treatment and reducing the discomfort of patients during treatment. Moreover, each sub-tube portion 113 formed by cutting is relatively independent and has excellent deformation performance, thereby enhancing the fit with the insert component 12 and the fit among the sub-tube portions 113, which facilitate reducing an overall outer diameter of the multi-cavity component 11. For example, the overall outer diameter of the multi-cavity component 11 can be within 3.2 mm, thereby improving the passability in the blood vessel and meeting requirements for use in smaller blood vessels.

In some embodiments, as shown in FIG. 1 and FIG. 2, the number of the electrodes 13 provided on each sub-tube portion 113 may be multiple. Optionally 2 to 4, and the specific number of the electrodes 13 can be set according to actual use requirements. Along the axial direction of the multi-cavity component 11, required electrodes 13 are arranged at intervals on each sub-tube portion 113. In this way, a discharge range of the electrodes 13 is increased in the axial direction, thereby increasing a treatment range. For example, the electrodes 13 disposed on the sub-tube portions 113 may be slipped onto the sub-tube portions 113 from a tip of the distal end thereof, and moved to be adjusted to set positions on the sub-tube portions 113; or can be formed by wrapping around the set positions on the sub-tube portions 113.

In one possible implementation scheme, as shown in FIG. 1, the insert component 12 may be a tube, such as an elongated filamentary structure, coaxially arranged with the multi-cavity component 11. One end of the insert component 12 is inserted from the central cavity 111 and extends towards the distal end 102 to be connected to the distal end of the multi-cavity component 11. For example, the distal end of the insert component 12 and the distal end of the multi-cavity component 11 are fixedly connected. A portion of the insert component 12 in the central cavity 111 is movable reciprocally in the central cavity 111 to achieve pulling the distal end of the multi-cavity component 11 to move, so that each sub-tube portion 113 can protrude outward into the curved shape, or form a cage shape with a reduced degree of curvature. Under the action of its own elastic restoring force of each sub-tube portion 113, the sub-tube portion 113 returns from a curved state to a non-curved state, and is in a horizontal free state (for example, a free state of extending straight along the length direction of the multi-cavity component 11 is presented). Moreover, a material of the insert component 12 may be the same as the material of the multi-cavity component 11, thereby improving the convenience of manufacturing. In one possible implementation scheme, as shown in FIG. 2, the insert component 12 may be made of metal wires such as stainless steel or nickel titanium, and the number of the insert components 12 can be determined according to the use requirements, for example, the number of insert components 12 ranges from 2 to 4, but is not limited herein. For example, the number of the insert components 12 shown in FIG. 2 is 3.

In some embodiments, the length directions of the 2 to 4 insert components 12 extend along a longitudinal axis direction of the multi-cavity component 11. The 2 to 4 insert components 12 are arranged adjacent to each other or spaced apart from each other. For example, the 2 to 4 insert components 12 are arranged side-by-side in a plane parallel to the longitudinal axis of the multi-cavity component 11, and the side-by-side arrangement can be closely attached to each other or arranged at intervals. Alternatively, the 2 to 4 insert components 12 are spaced apart along the circumferential direction of the multi-cavity component 11. Alternatively, along a cross-section direction of the multi-cavity component 11, the 2 to 4 insert components 12 are located at different cross-section positions. For example, when the number of the insert components 12 is at least three, a cross-section defined by the at least three insert components 12 is perpendicular to the length direction of the multi-cavity component 11.

It can be understood that a length direction of each of the 2 to 4 insert components 12 itself extends along the longitudinal axis of the multi-cavity component 11, which does not merely include that any part of each insert component 12 in the length direction itself extends along the longitudinal axis of the multi-cavity component 11, but also include that at least a part of each insert component 12 in the length direction itself extends along the longitudinal axis of the multi-cavity component 11 (for example, the length direction of the part of the insert component 12 adjacent to the sub-tube portion 113 extends along the longitudinal axis direction of the multi-cavity component 11), and at least a part of the insert component 12 in the length direction itself may be deviated from the central axis of the multi-cavity component 11 and be fixed to other structures (for example, an operating handle).

One end of the metal wire (i.e., the insert component 12) is inserted into the central cavity 111, and extends to the distal end 102 to connect to the distal end of the multi-cavity component 11, so that the distal end of the multi-cavity component 11 can be pulled to move, to achieve the transform of the sub-tube portions 113 as needed between the configuration of protruding outward in the curved shape and in the cage shape. In addition, a PTFE (polytetrafluoroethylene) or PI (polyimide) coating can be further provided on a surface of the metal wire such as stainless steel or nickel titanium, to improve the durability of the metal wire and allow the metal wire to have higher safety, thereby meeting the requirements of medical use.

In some embodiments, as shown in FIG. 1 and FIG. 4, each electrode 13 is connected to an insulated electrical lead 14 disposed in the peripheral cavity 112, through which current is delivered to each electrode 13. For example, an effective diameter of the insulated electrical lead 14 is not less than 0.12 mm, and an overall diameter of an ultra-fine multi-layer insulated electrical lead does not exceed 0.25 mm. The insulation breakdown strength of the insulated electrical lead 14 exemplarily reaches above 5 kV. Through a multi-layer insulation structure, the risk of electromagnetic interference generated during corona discharge is reduced, and the risk of short circuit caused by conduction during saline perfusion is also reduced. Simultaneously, a length of the sub-tube portions 113 is exemplarily set to 30 mm to 80 mm, so that the length of each sub-tube portion 113 is within an appropriate range. When in a curved shape, the sub-tube portions 113 may have diameter sizes that meet the practical requirements, thereby avoiding insufficient working area due to insufficient length or insufficient structural strength due to overlength.

In some embodiments, as shown in FIG. 4, the insulated electrical lead 14 includes a conductive core 141 and an insulating layer 142. The conductive core 141 is connected to the electrodes 13. The insulating layer 142 is configured as multi-layer structure, and the multiple layers of the insulating layers 142 are wrapped on the conductive core 141 layer by layer and extend along a length direction of the conductive core 141. Each insulating layer 142 is coaxially arranged with the conductive core 141. For example, the conductive core 141 may be composed of a copper core, and a diameter is exemplary not less than 0.12 mm. A PTFE (polytetrafluoroethylene) or PI (polyimide) coating may be used for insulation among ultra-fine copper wires composing the copper core, to improve the insulation ability of the conductive core 141. In the embodiment of the present disclosure, the insulating layer 142 may optionally be configured with four layers, such that under the premise of meeting the overall insulation performance requirements, the overall size of the insulated electrical lead 14 can pass through the corresponding peripheral cavity 112 (referring to FIG. 3).

In some embodiments, during a PFA (pulsed electric field ablation) surgery, intracardiac potential signals need to be mapped to achieve electrophysiological examination and immediate efficacy assessment of ablation therapy. Mapping is carried out by amplifying and acquiring weak cardiac electrical signals, while ablation requires the release of high-voltage pulse energy through the electrodes. In order to prevent the high-voltage pulse from damaging a detection circuit of the cardiac electrical signals, in the present disclosure, each electrode 13 is connected to individual lead wire, and an insulation voltage between any two electrodes 13 can exemplarily reach above 5 kV, thereby integrating mapping and ablation functions, and achieve multiplexing of the ablation and mapping functions through fast switching in a host system.

In some embodiments, as shown in FIG. 1 and FIG. 3, a braided layer 114 is provided in the multi-cavity component 11. The braided layer 114 extends from the proximal end towards the sub-tube portions 113, approaching the sub-tube portions 113 but does not extend into a region of the sub-tube portions 113. In a radial direction, the braided layer 114 is provided on an outer side of each of the peripheral cavities 112. For example, in the radial direction, for a region of each peripheral cavity 112 located on proximal sides of the sub-tube portions 113, the braided layer 114 is provided on the outer side of each peripheral cavity 112.

By providing the braided layer 114, a torque transmission capability of the multi-cavity component 11 is improved, thereby achieving reliable movement of the whole component in the blood vessel. The braided layer 114 may be a stainless steel braided mesh, exhibiting high strength and suitable elastic bending deformation performance. The braided layer 114 may be disposed in the multi-cavity component 11 in a discontinuous way in the circumferential direction, instead, a plurality of braided layers 114 are respectively arranged at the outsides of the peripheral cavities 112, and the braided layers 114 are not connected with each other. In this way, the strength of the peripheral cavities 112 is enhanced and the overall structural strength is improved. In addition, since the multi-cavity component 11 adjacent to the distal end needs to be cut to form the sub-tube portions 113, the braided layer 114 does not extend into the region of the sub-tube portions 113, avoiding affecting the cutting process of the multi-cavity component 11.

In some other embodiments, as shown in FIG. 3, the braided layer 114 may be arranged by surrounding the multi-cavity component 11 in the circumferential direction, with each peripheral cavity 112 is located in a region surrounded by the braided layer 114. In this configuration, in the circumferential direction of the multi-cavity component 11, the outer sides of the sub-tube portions 113 are surrounded by the same braided layer 114, which simultaneously provides protection for the sub-tube portions 113 to avoid accidental puncture.

In some embodiments, to achieve adjustment of the hardness of the multi-cavity component 11 as needed, it may be achieved by changing a material forming the multi-cavity component 11, and it may also be achieved by changing a thickness or a density of the braided layer 114, diverse setting ways are allowed.

In some embodiments, in a direction perpendicular to the length of the multi-cavity component 11, cross-sectional shapes of the peripheral cavities 112 are at least partially identical. That is, the cross-sectional shapes of the peripheral cavities 112 obtained in a same reference direction are at least partially identical. The insulated electrical lead 14 extends out of the peripheral cavities 112. In this way, there is no need to selectively insert the insulated electrical lead 14 due to the different shapes of the peripheral cavities 112, thereby improving the convenience of installation. Moreover, inner wall surfaces of the peripheral cavities 112 are configured as smooth curved surfaces with a small friction resistance, which is conducive to increasing the smoothness of moving the insulated electrical lead 14, and also facilitates the flow of fluids (such as saline). For example, it may provide the inner wall surfaces of the peripheral cavities 112 as the smooth curved surfaces by a processing technology, or by providing a PTFE (polytetrafluoroethylene) lining layer. Similarly, the inner wall surface of the central cavity 111 may also be provided with the PTFE (polytetrafluoroethylene) lining layer to improve the surface smoothness.

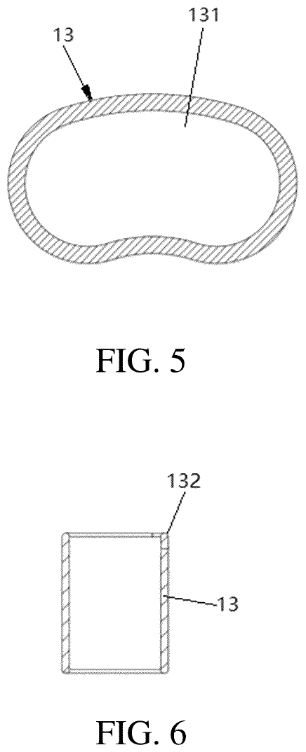

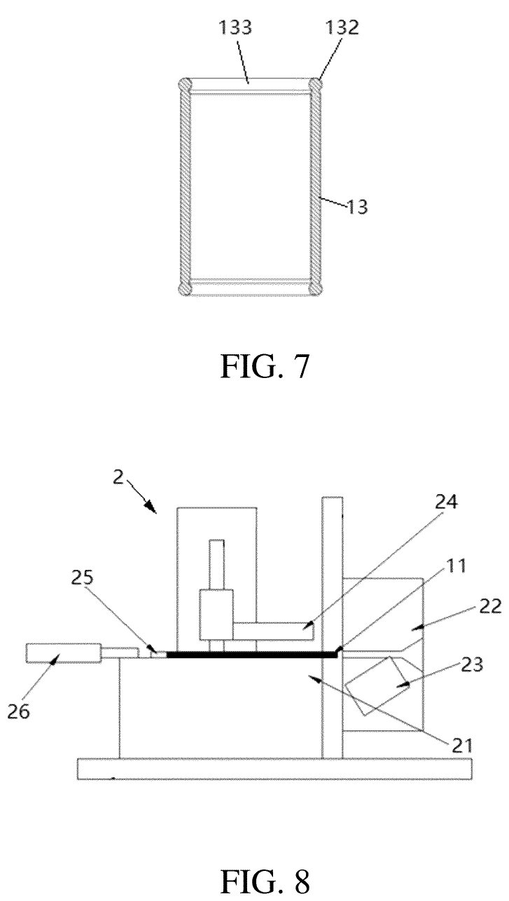

In some embodiments, as shown in FIG. 5 to FIG. 7, a receiving hole 131 is defined in each electrode 13 (referring to FIG. 1), and the receiving hole 131 is used to be inserted with the respective sub-tube portion 113, so that the electrodes 13 can be mounted on the sub-tube portions 113. In addition, a discharge side of each end of the electrodes 13 is provided with a voltage balancing structure 132. Alternatively, the discharge side of each end of the electrodes 13 is connected with a voltage balancing ring 133, and the voltage balancing structure 132 is provided on the voltage balancing ring 133. For example, since the pulse energizing device 1 needs to withstand a higher voltage, during pulse electric field ablation surgery, the high-voltage pulse energy is discharged through electrodes on the catheter. When the high-voltage pulse is discharged, in order to prevent tip discharge or spark discharge on the electrodes, the electric field distribution is designed to be more uniform. In the embodiment of the present disclosure, by providing the voltage balancing structures 132 or the voltage balancing rings 133 having the voltage balancing structures 132 on the discharge sides at both ends of the electrodes 13, and the voltage balancing structures 132 provided with smooth circular arc curved surfaces, the discharge sides at both ends of the electrodes 13 are free of tips due to the voltage balancing structures 132. Therefore, the electric field distribution is more uniform, and no serious power line distortion point is formed at both ends of the electrodes 13, avoiding or reducing the spark discharge caused by the tips of the electrodes 13 when a high-voltage nanosecond pulse exemplarily reaching 10 kV, thereby improving the safety and service life.

In some embodiments, as shown in FIG. 1 and FIG. 5, in a direction perpendicular to the length of the sub-tube portion 113, at least a cross-sectional shape of the receiving hole 131 is identical to cross-sectional outer contour shapes of the sub-tube portions 113, and the electrodes 13 are conforming to outer walls of the sub-tube portions 113. In this way, after the electrodes 13 are installed on the sub-tube portions 113, the electrodes 13 can be closely conformed to outer surfaces of the sub-tube portions 113, which not only achieves discharge reliably, but also forms no local protrusion, thereby reducing the overall radial dimension of the multi-cavity component 11. In addition, in this way, cross-sectional outer contour shapes of the electrodes 13 may be identical to or different from the cross-sectional shape of the receiving hole 131, and this configuration can be adjusted as needed.

In some embodiments, in the direction perpendicular to the length of the sub-tube portion 113, the cross-sectional outer contour shapes of the electrodes 13 are identical to the cross-sectional outer edge shapes of the sub-tube portions 113. This configuration can ensure that cross-sectional shapes of the multi-cavity component 11 perpendicular to the axial direction remain consistent and are all circular, the overall aesthetics are excellent, and it is also convenient to be delivered through the blood vessel.

In some embodiments, a positioning sensor (not shown in the figure) for positioning is provided on at least one of the sub-tube portions 113, and the positioning sensor is adjacent to the proximal end of the multi-cavity component 11. In this way, positions of the sub-tube portions 113 can be sensed by the positioning sensor, thereby achieving accurate treatment. Optionally, the positioning sensor is a magnetic positioning sensor, which has an excellent positioning effect and is safe in operation.

The pulse energizing device 1 provided by the embodiment of the present disclosure is provided with the multi-cavity component 11, which can be processed directly on the basis of the multi-cavity component 11 without requiring too many other complicated structural designs, resulting in a simpler structure of the pulse energizing device 1. The processing and production are achieved through cutting. The production process is relatively simple with low requirements, and can be reproduced repeatedly, which excellently meets the reproducibility requirements of the pulse energizing device 1. Through a multi-layer insulation design of the insulated electrical lead 14, the insulation capacity between electrodes 13 is improved, so that insulation levels of different electrodes 13 can meet nanosecond pulse discharge requirements with higher voltage and higher repetition frequency, thereby preventing the generation of corona discharge and interfering electrical signals. Through the design of the voltage balancing structures 132 at the end surfaces of the electrodes 13, the electric field distribution is more uniform, thereby effectively preventing the generation of spark discharge and greatly reducing the heat generation during the discharge process. Through a form-fitting design between the electrodes 13 and the sub-tube portions 113, the overall outer diameter of the multi-cavity component 11 is effectively reduced, thereby improving the overall deliverability and passability. In addition, each electrode 13 is connected to a separate insulated electrical lead 14, and separate insulated electrical leads 14 are insulated from each other, thereby realizing the time-sharing multiplexing of the electrodes 13. Therefore, during PFA surgery, ablation and mapping can be performed at different periods based on the same electrode 13.

In one embodiment of the present disclosure, a processing method is further provided to process the pulse energizing device 1. As shown in FIG. 8, the processing method employs a processing tooling 2 to process the multi-cavity component 11. The processing tooling 2 includes a workbench 21, a blade holder 22 mounted on the workbench 21, a cutting blade 23 mounted on the blade holder 22, a positioning block 24 configured to position the multi-cavity component 11, a positioning seat 25 configured to abut against the multi-cavity component 11, and a push rod 26 configured to push the positioning seat 25. The processing method includes steps as follows.

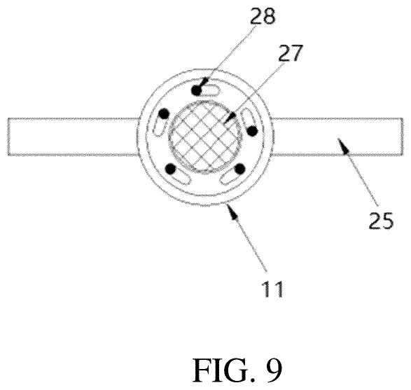

As shown in FIG. 8 and FIG. 9, a positioning pin 27 is inserted into the central cavity 111 of a multi-cavity component 11 to be processed, and a core rod 28 is inserted into a respective peripheral cavity 112. The positioning pin 27 and the core rod 28 are used to improve the overall hardness of the multi-cavity component 11 during processing, thereby preventing deformation during processing. Then, the multi-cavity component 11 is placed on the workbench 21 of the processing tooling 2 with one end abutting against the positioning seat 25 and the middle position of the multi-cavity component 11 being pressed by the positioning block 24 for positioning to prevent the multi-cavity component 11 from warping during the moving cutting. After the multi-cavity member 11 is positioned, the cutting blade 23 on the processing tooling 2 is brought into abut against a part of the multi-cavity component 11 to be cut. After that, the positioning seat 25 is driven to move by a driving force applied by the push rod 26, causing the multi-cavity component 11 to move toward the cutting blade 23, so that the multi-cavity component 11 is cut by the cutting blade 23, to form the sub-tube portions 113.

Certainly, in some other embodiments, the multi-cavity component 11 may also be prevented from moving by the push rod 26 abutting against the positioning seat 25, and then the cutting blade 23 is moved toward the multi-cavity component 11 to cut, thereby forming the sub-tube portions 113. After the cutting is completed, the positioning block 24 is released, the processed multi-cavity component 11 is removed from the processing tooling 2, and the positioning pin 27 and each core rod 28 are respectively pulled out.

In some embodiments, during cutting the multi-cavity component 11, the number of the cutting blade 23 is equal to the number of the sub-tube portions 113 to be formed by cutting. An angle of the cutting blade 23 relative to an axial direction of the multi-cavity component 11 is adjusted, so that the cutting blade 23 is inclined at a certain angle relative to the axial direction of the multi-cavity component 11, thereby reducing a cutting resistance and the abrasion of the cutting blade 23.

In some other embodiments, an angle between the cutting blade 23 and the positioning block 24 may further be adjusted, so that a pressure is generated by a spring after the cutting blade 23 is installed on the blade holder 22, to ensure that the cutting blade 23 always abuts against a wall of the blade holder 22 by a lateral force, thereby ensuring the position accuracy of the cutting blade 23.

In some embodiments, during cutting the multi-cavity component 11, the cutting blade 23 may continuously cut along a direction from a tip of the distal end 102 of the multi-cavity component 11 toward the proximal end to form the sub-tube portions 113, and a cutting length is exemplarily 30 mm to 80 mm. In this way, the distal end of each sub-tube portion 113 is a free end. Then, the electrodes 13 are mounted onto the sub-tube portions 113 respectively from the ends of the sub-tube portions 113, so that each sub-tube portion 113 is provided with the electrodes 13. Each electrode 13 is connected to the insulated electrical lead 14. The insulated electrical lead 14 extends through the peripheral cavity 112 of a sub-tube portion 113 on which the electrodes 13 are located, and then extends to the proximal end of the multi-cavity component 11 until it can be connected to the high-voltage pulse generator.

In some embodiments, during cutting the multi-cavity component 11, the cutting blade 23 may continuously cut from a position away from the tip of the distal end by a preset distance in a direction toward the proximal end to form the sub-tube portions 113, and the cutting length is 30 mm to 80 mm. For example, in this cutting method, the cutting blade 23 does not start to cut directly from the tip of the distal end, but from a position spaced from the tip of the distal end by the preset distance, and the preset distance may be between 5 mm and 15 mm, to ensure that the distal ends of the sub-tube portions 113 formed after cutting are not dispersed. Of course, the preset distance may also be other ranges. After the cutting is completed and the sub-tube portions 113 are formed, each sub-tube portion 113 is respectively wrapped with the electrodes 13. Each electrode 13 is connected to the insulated electrical lead 14. The insulated electrical lead 14 extends through the peripheral cavity 112 of the sub-tube portion 113 on which the electrodes 13 are located, and then extends to the proximal end of the pulse energizing device 1 until it can be connected to the high-voltage pulse generator. In one embodiment, after the electrodes 13 are arranged, one end of the insert component 12 extends through the central cavity 111 of the multi-cavity component 11 and extends to fixedly connect with the distal end of each sub-tube portion 113 to form an integral structure. Therefore, the sub-tube portions 113 can be transformed between two configurations of extending along a straight line and protruding outward in a curved shape by pulling the insert component 12.

In some other embodiments, when a manner of starting to the cut from the tip of the distal end of the multi-cavity component 11 is employed, after the electrodes 13 are arranged on each sub-tube portion 113, one end of the insert component 12 may extend through the central cavity 111 of the multi-cavity component 11, and then the free end of each sub-tube portion 113 is connected to the distal end of the insert component 12, thereby completing the manufacturing process.

In some other embodiments, when a manner of the cutting blade 23 starting to continuously cut from a position away from the tip of distal end of the multi-cavity component 11 by the preset distance toward the proximal end of the pulse energizing device 1 is employed, firstly, one end of the insert component 12 may extend through the central cavity 111 of the multi-cavity component 11 and be bonded or fused integrally with the distal end of the multi-cavity component 11; then, the cutting is performed to form the sub-tube portions 113. Thus, the processing method is versatile and highly flexible.

In the processing method provided by the embodiments of the present disclosure, the positioning pin 27 is first inserted into the central cavity 111, and the core rods 28 are respectively inserted into the peripheral cavities 112; then the multi-cavity component 11 to be cut is placed on the processing tooling 2 and cut to form the sub-tube portions 113; and then, the electrodes 13 are provided on the sub-tube portions 113 respectively, and the insert component 12 is connected in such a manner that the distal end of the insert component 12 is connected to the distal end of the multi-cavity component 11. In this way, the production and processing of the pulse energizing device 1 are completed. The processing method achieves processing and production by cutting, and the processing method has relatively simple processes with low requirements. Moreover, the processing method has excellent repeatability, thereby well meeting the reproducibility requirements of the pulse energizing device 1.

The above descriptions are merely the specific embodiments of the present disclosure, but the protection scope of the present disclosure is not limited thereto. Any changes, variations, equivalent replacements, and improvements made by those skilled in the art without departing from the spirit and scope of the present disclosure should fall into the protection scope of the claims of the present disclosure. Therefore, the protection scope of the present disclosure should be based on the protection scope of the claims.

1. A pulse energizing device, comprising:
a multi-cavity component, defining a central cavity and a plurality of peripheral cavities surrounding the central cavity; the plurality of peripheral cavities and the central cavity extending along an axial direction without communicating with each other; wherein a portion of the multi-cavity component between a proximal end and a distal end thereof in the axial direction is cut along the axial direction into a plurality of sub-tube portions that are separated in a circumferential direction; each of the plurality of sub-tube portions is provided with a respective peripheral cavity therein, and each of the plurality of sub-tube portions is provided with a through hole communicating with the corresponding peripheral cavity therein; and the distal end of the multi-cavity component is movable along the axial direction of the multi-cavity component, to drive the plurality of sub-tube portions to transform between one configuration of extending along a straight line and another configuration of protruding outward in a curved shape;
at least one insert component, one end of the at least one insert component extending through the central cavity to connect with the distal end of the multi-cavity component; and
a plurality of electrodes, each of the plurality of electrodes being mounted around a respective sub-tube portion and configured to deliver a pulse current.

2. The pulse energizing device of claim 1, wherein a wall thickness of the multi-cavity component is greater than a preset value; and the preset value is 0.5 mm to 1.5 mm.

3. The pulse energizing device of claim 1, wherein the plurality of electrodes are configured to deliver the pulse current when the plurality of sub-tube portions are in the another configuration of protruding outward in the curved shape or in the configuration of extending along the straight line.

4. The pulse energizing device of claim 1, wherein the at least one insert component is a metal wire; the at least one insert component comprises a plurality of insert components, and a number of the plurality of insert components is 2 to 4; and the plurality of insert components with the number of 2 to 4 are arranged in contact with each other or spaced apart from each other.

5. The pulse energizing device of claim 1, wherein each of the plurality of electrodes is connected to an insulated electrical lead disposed in the peripheral cavity of the respective sub-tube portion, and a length of each of the plurality of sub-tube portions is 30 mm to 80 mm.

6. The pulse energizing device of claim 5, wherein the insulated electrical lead comprises a conductive core and a plurality of insulating layers, the conductive core is connected to a corresponding electrode; the plurality of insulating layers are wrapped on the conductive core layer by layer and extend along a length direction of the conductive core; and the plurality of insulating layers are coaxially arranged with the conductive core.

7. The pulse energizing device of claim 1, wherein a braided layer is provided in the multi-cavity component, the braided layer extends from the proximal end of the multi-cavity component toward the plurality of sub-tube portions, approaching the plurality of sub-tube-portions without extending into a region of the plurality of sub-tube portions.

8. The pulse energizing device of claim 7, wherein in a radial direction, the braided layer is provided on an outer side of each of the plurality of peripheral cavities.

9. The pulse energizing device according to claim 8, wherein in the radial direction, for a region of each of the plurality of peripheral cavities located on a proximal side of the plurality of sub-tube portions, the braided layer is provided on the outer side of each of the plurality of peripheral cavities.

10. The pulse energizing device of claim 7, wherein the multi-cavity component is surrounded by the braided layer in the circumferential direction, and each of the plurality of peripheral cavities is located in a region surrounded by the braided layer.

11. The pulse energizing device of claim 1, wherein in a direction perpendicular to a length of the multi-cavity component, cross-sectional shapes of the plurality of peripheral cavities are at least partially identical; and an inner wall surface of each of the plurality of peripheral cavities is a smooth curved surface.

12. The pulse energizing device of claim 1, wherein each of the plurality of electrodes is provided with a receiving hole, and the receiving hole is configured to be inserted with the respective sub-tube portion; and
wherein a discharge side of each of two ends of each of the plurality of electrodes is provided with a voltage balancing structure or a voltage balancing ring provided with the voltage balancing structure.

13. The pulse energizing device of claim 12, wherein in a direction perpendicular to a length of the plurality of sub-tube portions, a cross-sectional shape of each receiving hole is identical to a cross-sectional outer contour shape of the respective sub-tube portion, and each of the plurality of electrodes is mounted around an outer wall of the respective sub-tube portion.

14. The pulse energizing device of claim 1, wherein in a direction perpendicular to a length of the plurality of sub-tube portions, a cross-sectional outer contour shape of each of the plurality of electrodes is identical to a cross-sectional outer contour shape of the respective sub-tube portion.

15. The pulse energizing device of claim 1, wherein at least one of the plurality of sub-tube portions is provided with a positioning sensor that is configured to position, and the positioning sensor is adjacent to the proximal end of the multi-cavity component.

16. A processing method for processing the pulse energizing device as claimed in claim 1, the processing method comprising:
inserting a positioning pin into a central cavity of a multi-cavity component to be processed, and inserting a plurality of core rods into a plurality of peripheral cavities respectively;
placing and fixing the multi-cavity component to be processed on a processing tooling, and bringing a cutting blade on the processing tooling to abut against a portion of the multi-cavity component to be cut;
driving the multi-cavity component or the cutting blade on the processing tooling to move, causing the cutting blade to cut the multi-cavity component to form the plurality of sub-tube portions; and
removing the multi-cavity component from the processing tooling, and pulling out the positioning pin and each core rod respectively.

17. The processing method of claim 16, wherein the cutting blade continuously cut along a direction from a tip of the distal end of the multi-cavity component toward a proximal end to form the plurality of sub-tube portions, and a first cutting length is 30 mm to 80 mm; or,
wherein the cutting blade continuously cut from a position away from the tip of the distal end by a preset distance in a direction toward the proximal end to form the plurality of sub-tube portions, and a second cutting length is 30 mm to 80 mm.

18. The processing method of claim 17, wherein each of the plurality of sub-tube portions is mounted with at least one electrode; and each electrode is connected to an insulated electrical lead, and the insulated electrical lead extends through the peripheral cavity of a respective sub-tube portion on which the electrode is located.

19. The processing method of claim 17, wherein each of the plurality of sub-tube portions is wrapped with at least one electrode; and each electrode is connected to the insulated electrical lead, and the insulated electrical lead extends through the peripheral cavity of the respective sub-tube portion on which the electrode is located.

20. The processing method of claim 19, wherein one end of the at least one insert component is inserted into the central cavity of the multi-cavity component and integrally connected to the distal end.
